(19) United States
(12) Patent Application Publication
(10) Pub. No.: US 2005/0049591 A1
(43) Pub. Date: Mar. 3, 2005

(54) Device for reduction of a vertebra

(76) Inventors: Mark Kapitov, Haifa (IL); Alexander Kalenkovich, Haifa (IL)

Correspondence Address: Kalenkovich Alexander, Winner Joseff St. 10/2, Haifa, 32493, IL

(21) Appl. No.: 10/858,429

(22) Filed: Jun. 2, 2004

(30) Foreign Application Priority Data
Aug. 28, 2003 (IL) 157618

(51) Int. Cl. A61B 17/58

(52) U.S. Cl. 606/61

(57) ABSTRACT

This invention relates to the area of medical technique and, particularly, to orthopedic devices. The proposed device intended to perform reduction of a vertebra by elevation of the pressure in the abdominal cavity this pressure brings the vertebra into its anatomically correct position. As a result, such device allows to treat spodylolysis and spondylolisthesis without surgical intervention.

### CROSS REFERENCE APPLICATION

This patent application claims the benefit of the earlier filed Israeli Patent Application Ser. No. filed on

### BACKGROUND OF THE INVENTION

This invention relates to the area of medical orthopedic devices, particularly, to bandages and orthopedic corsets.

There is description of a back support device (U.S. Pat. No. 4,245,628) that used for treatment of some back diseases. However, such bandages and orthopedic corsets provide only support of a vertebral column and, in such a way, they present only a prophylactic means for prevention of diseases of a vertebral column.

In order to treat without surgical intervention such diseases of a vertebral column, as spondylolysis and spondylolisthesis, this invention proposes a device of reduction of a vertebra in its anatomically correct state, the device provides elevation of pressure in the abdominal cavity, that ensures rehabilitation of a tela conjunctiva in region of a fissure or fracture, and eliminates the phenomenon of radiating pain in legs.

### BRIEF SUMMARY OF THE INVENTION

An aim of this invention is to construct a device for reduction of a vertebra; such device allows to perform non-surgical treatment of some diseases of a vertebral column by bringing the injured vertebra into its anatomically correct state. This treatment is based on elevation of pressure in the abdominal cavity, when this pressure is directed toward the vertebra.

The proposed device includes a pressing unit with an applicator; the surface of this applicator, which should be in immediate contact with an abdomen, has the spherical or another cambered shape, this provides elevation of pressure in the abdominal cavity and, in such a way, reduction of the injured vertebra.

In addition, the device comprises a back support, which is constructed from two links situated from both sides of the vertebral column with a possibility of adjustment of spacing interval between these links in vertical and horizontal directions. This eliminates pressure on a vertebral column and makes difficult to a patient to bend. This, in turn, prevents anterior shifting the injured vertebra and, as result, aggravation of pain in the vertebra and radiating pain in the legs.

The proposed orthopedic device includes following details:
a pressing unit with a joined applicator; the surface of this applicator, which is intended to be in immediate contact with the abdomen, has spherical or another cambered shape of a plane; a back support, which constructed from two links, disposed from both sides of the vertebral column with means for adjustment of spacing between these links in horizontal direction in order to fit the device to a user's figure.

The present invention will be understood more fully from the detailed description given herein below and from the accompanying drawings of the preferred embodiment of the invention, which, however, should not be construed as exhaustive to the invention but are for explanation and understanding only.

### BRIEF DESCRIPTION OF THE SEVERAL VIEWS OF THE DRAWING

The essence of the invention is illustrated by drawings, wherein:

FIG. 1 is a general plan view of the device for reduction of a vertebra.

FIG. 2 is a general view of the applicator without a cover.

FIG. 3 is cross-section 3-3 of the link of the back support, which is shown in FIG. 1.

### DETAILED DESCRIPTION OF THE INVENTION

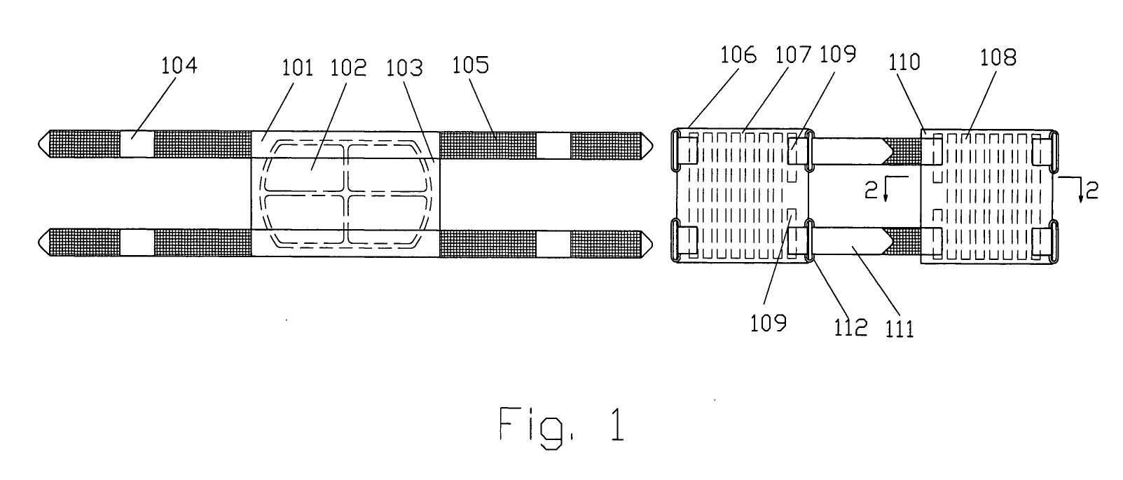

FIG. 1 shows the general plan view of the device for reduction of a vertebra. The device comprises: a pressing unit 101, which, in turn, consists of applicator 102 installed in cover 103; fixing belts 104 with a “Velcro” locking units 105; a back support 106 constructed from two links 107, each aforementioned link is provided with a set of stays 108 and 109 from elastic material, for example, polymer, these stays are arranged in cover 110. Such construction ensures fitting the links with the user's figure.

Stays 109 are placed with such distance between them in vertical direction, that it provides freely motion of the patient's body and, at the same time, obviates additional loading on the soft tissues of the support-motor system in the area of the waist and elevation of mechanical pressure in the aforementioned soft tissues; this, in turn, prevents formation of local anemia.

One aforementioned link is furnished with belts 111 with “Velcro” locking units similar to the locking units on the belts of the pressing unit 101, and the second link has cramps 112, the belts is passing through this cramps for fastening the device. Besides, the similar cramps are installed on the external side of the links for tightening pressing unit 101 and locking by belts 103. Similar cramps are fastened on the ends of the links; they serve for tightening and locking by belts 103.

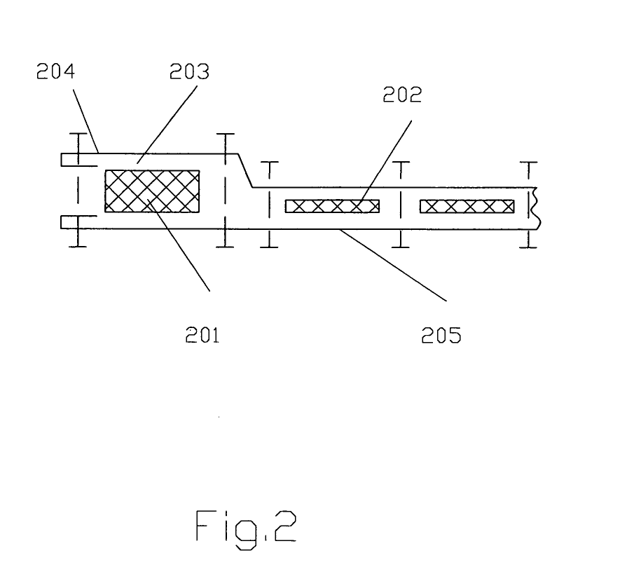

FIG. 2 shows stays 201 and 203, which are sawn in pocket 203 of cover 204 of link 205, and stays 201, which are situated at minimum distance from a vertebral column, have larger thickness; it prevents pressure on a vertebral column.

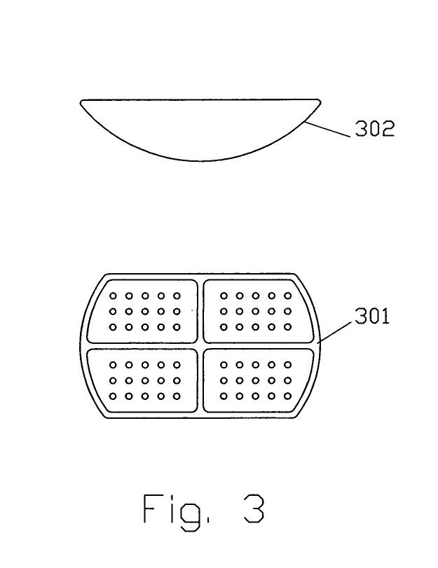

As shown in FIG. 3, applicator 301 has the spherical are another cambered shape of its surface 302, which should be in immediate contact with an abdomen, this provides in the process of dressing the device by a patient indentation of the muscles of an abdomen and, therefore, elevation of abdominal pressure to such a point, which provides reduction of an injured vertebra and effective formation of a callus.

The device operates in the following manner. The device is dressed on the patient, when he lies with his abdomen downwards on the pressing unit with maximum relaxation of the abdominal muscles.

In doing so, the pressure unit causes maximum deflection of the abdominal muscles and provides elevation of the pressure in the abdominal cavity. Then the back support, which was preliminary adjusted in its width, is positioned on the patient's back and the front lock is tightened by the belts toward the back support and should be locked by the “Velcro” units. In doing so, the abdominal pressure brings the vertebral column in its anatomical correct state, this ensures effective formation of a callus and prevents radiating pain in the legs because moderation of pressure on the nervous roots.

The device for reduction of a vertebra can be used for therapy of spondylolysis, spondylolisthesis without surgical intervention. Besides, the device for reduction of a vertebra provides possibility of normal activity of a patient during the treatment period.

Although the invention has been described and illustrated with a certain degree of particularity, it is understood that the present disclosure has only been made by way of example, and that various modifications thereof may be resorted to by those skilled in the art without departing from the spirit and scope of the invention, as hereinafter claimed.

1. A device for reduction of a vertebra consisting of:
a pressing unit, which includes an applicator; the surface of said applicator, which should be in immediate contact with an abdomen, has spherical or other cambered shape, this provides elevation of pressure in the abdominal cavity in contact said applicator with muscles of the abdomen;
means for fixing said pressing unit of a patient's body;
a back support, which is constructed from two links with such joining elements that they gives possibility of adjustment of the distance between said links throughout the width; said stays belonging to said link, which is nearest to the vertebral column, are arranged with such mutual distance, that this provides freely motion of a patient.

2. The device for reduction of a vertebra according to claim 1, wherein said links of said back support allow to adjust the device on the patient with possibility to fit it for features of said patient's figure.

3. The device for reduction of a vertebra according to claim 1, wherein said pressing unit has from its each side fastening belts and locking means, which allow fixation of said pressing unit on said patient's body.

4. The device for reduction of a vertebra according to claim 2, wherein the distance between said links in horizontal direction obviates immediate contact said links with a vertebral column.

5. The device for reduction of a vertebra according to claim 5, wherein said stays has different width, and width of said stays, which are situated near the vertebral column, has higher magnitude.
